(19) United States
(12) Patent Application Publication
(10) Pub. No.: US 2025/0235118 A1
(43) Pub. Date: Jul. 24, 2025

(54) MR Coil for Intraoral MR Measurements

(71) Applicant: Siemens Healthineers AG, Forchheim (DE)

(72) Inventors: Antonia Huber, Karlsruhe (DE); Matthias Korn, Moehrendorf (DE); Matthias Malzacher, Nürnberg (DE)

(73) Assignee: Siemens Healthineers AG, Forchheim (DE)

(21) Appl. No.: 19/027,582

(22) Filed: Jan. 17, 2025

(30) Foreign Application Priority Data
Jan. 18, 2024 (DE) 10 2024 200 458.8

(51) Int. Cl. A61B 5/055; A61B 5/00; G01R 33/34

(52) U.S. Cl. CPC A61B 5/055; A61B 5/004; A61B 5/4542; G01R 33/34046; G01R 33/34084

(57) ABSTRACT

The disclosure relates to an MR coil for intraoral MR measurements on a target section in the dental area. The target section may include at least part of a dental arch. The MR coil May have coil conductors which form at least one resonant circuit. The MR coil may have a central part and two legs protruding from the central part in which coil conductors of the resonant circuit are arranged. The MR coil can be placed on the dental arch in such a way that the legs encompass the dental arch such that one leg is arranged on the inside and one leg on the outside of the dental arch. The MR coil may have a shape adapted to the shape of the target section.

### CROSS REFERENCE TO RELATED APPLICATIONS

This patent application claims priority to German (DE) patent application Ser. No. 102024200458.8, filed Jan. 18, 2024, which is incorporated herein by reference in its entirety.

### BACKGROUND

### Field

The disclosure relates to a magnetic resonance coil (MR coil) for intraoral MR measurements in the dental area and an MR coil assembly.

### Related Art

There is a diagnostic interest in further developing magnetic resonance imaging (MRI) of teeth, jaws and gums in order, for example, to be able to better show inflammatory processes in the gums and in the roots of the teeth. MR measurements in the dental area place high demands on the local receiver coils due to the very fine structure of the teeth and roots of the teeth. In order to obtain a sufficiently high-resolution MR image, on the one hand the signal-to-noise ratio (SNR) must be very strong, especially at the roots of the teeth. On the other hand, it is advantageous to be able to show not just a single tooth, but a larger section of the jaw or an entire half of the jaw as it is not always clear in advance which tooth or which teeth are affected by the disease process. Furthermore, the area surrounding the affected tooth is also required for adequate diagnostics, and it is also advantageous to be able to display the jaw as a whole. Thus, by imaging a larger section of the jaw or one half of the jaw, the measurement time can be significantly reduced, thereby increasing patient comfort and patient throughput.

The article “Intraoral Approach for Imaging Teeth Using the Transverse B1 Field Components of an Occlusally Oriented Loop Coil,” Magnetic Resonance in Medicine, 72:160-165 (2014) describes a flat MR coil which a patient can hold between the occlusal surfaces in the mouth by clenching their teeth. However, this coil has the disadvantage that it is at a relatively large distance from the roots of the teeth because it rests on the occlusal surfaces of the teeth. Thus, the penetration depth of the coil is limited. As the coil axis is aligned in the z-direction of the MRI device, only the stray field oriented in the x-direction and y-direction contributes to the MR signal. In addition, the coil is connected to the MRI device via cables, which can have a negative impact on handling and patient comfort.

An RF coil (radio frequency coil) for MR measurements of a target volume in an oral area of an object to be examined is known from DE 10 2014 220 116 A1. However, only a single tooth or very few teeth can be observed with this RF coil due to the design. The preferred diameter is 2-3 cm.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a perspective view of an MR coil according to an embodiment of the disclosure;

FIG. 2 is a perspective view of the coil conductors of an MR coil according to an embodiment of the disclosure, mounted on a dental arch;

FIG. 3 is a perspective top view of the coil conductors of an MR coil of FIG. 2;

FIG. 4 is an illustration of the current path of a double quadrant coil according to an embodiment;

FIG. 5 is an illustration of the current path of a double quadrant coil according to an embodiment;

FIG. 6 is a view through a jaw with a double quadrant coil and marked B1 field lines;

FIG. 7 is a schematic view of a Transmission Line Resonator (TLR);

FIG. 8 is a perspective view of the coil conductors of a dental coil, which is configured as a TLR butterfly coil;

FIG. 9 is a perspective view of the dental coil of FIG. 8, mounted on a row of teeth;

FIG. 10 is a perspective view of the coil conductors of a dental coil, which is designed as a TLR;

FIG. 11 is an enlarged section of the coil conductors of FIG. 10 at the crossing point; and

FIG. 12 is a perspective view of the coil conductors of a dental coil, which is designed as a TLR butterfly coil with a common center bar.

The exemplary embodiments of the present disclosure will be described with reference to the accompanying drawings. Elements, features and components that are identical, functionally identical and have the same effect are—insofar as is not stated otherwise-respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure.

An object of the disclosure is to provide an MR coil for MR measurements in the dental area which overcomes the limitations of known MR coils. In particular, it is an object of the disclosure to provide an MR coil for intraoral MR measurements with which the widest possible area can be examined with an improved signal-to-noise ratio.

The disclosure achieves this object by means of an MR coil and an MR coil assembly according to one or more exemplary embodiments.

The disclosure provides an MR coil for intraoral MR measurements on a target section in the dental area, the target section comprising at least a part of a dental arch. The MR coil has coil conductors which form at least one resonant circuit. Furthermore, the MR coil has a central part and two legs protruding from the central part, in which coil conductors of the at least one resonant circuit are arranged. The MR coil may be designed to be placed on the dental arch in such a way that the legs encompass the dental arch and possibly the jaw in the target section in such a way that one leg may be arranged on the inside and one leg on the outside of the dental arch, the MR coil having a shape adapted to the shape of the target section.

The coil according to the disclosure may be characterized on the one hand by the fact that it encompasses the dental arch with the two legs in such a way that the coil conductors arranged in the legs come to lie very close to the roots of the teeth. This means that an MR measurement with a very high signal-to-noise ratio can be carried out on the roots of the teeth, on the gums and, if necessary, in the jaw. Furthermore, the coil may be so long that an area of several teeth can be recorded by one measurement. To achieve this, the shape of the MR coil may be adapted to the target section, which may comprise at least part of a dental arch. The MR coil may have the shape of a dental brace. This makes it possible to record a large area of the jaw, for example, a quadrant of the jaw, the entire lower jaw or upper jaw, or even the entire jaw (lower jaw and upper jaw) with the MR coil simultaneously in one MR measurement, for example to create an MR image of the entire quadrant of the jaw, half of the jaw or jaw. At the same time, the MR coil is comfortable to wear as a result of its shape, which may be adapted to the target section. During an MR measurement, it can simply be inserted into the mouth and placed over the teeth. As a result of its shape, which may be adapted to the dental arch, the patient does not have to hold it in place specifically by means of biting. On the other hand, especially with an inductively coupled MR coil, the patient can close their mouth and does not find the MR coil more obtrusive than a dental brace.

The MR coil may be adapted to the shape of the target section, the target section comprising at least a part of a dental arch. Thus, the MR coil may have an arch shape adapted to the dental arch. However, the adapted shape can also be adapted to a straight section of the dental arch, e.g. in the area of the molars. For example, the legs on the inside and outside of the dental arch can be shaped differently. The MR coil may have a standardized shape, i.e. the shape may be generally adapted to the shape of a standard dental arch, but not to the individual dentition of a patient. However, it is possible to provide a set of several MR coils according to the disclosure in different sizes in order, for example, to be able to cover different sizes of patient dentition, e.g. a child size and 2-3 different sizes for adult patients.

The MR coil is particularly suitable for intraoral MR measurements on humans, such as for diagnostic MR imaging in the dental area. The target section in the dental area may comprise at least part of a dental arch. The terms “dental area” and “dental arch” are to be understood broadly in this context. In particular, the term “dental arches” refers to the two horseshoe-shaped rows of teeth in humans, which are located in the upper and lower jaw. In a healthy set of teeth, a dental arch consists of 16 teeth. The dental arches are supported by the jaw. The target section can therefore also include the section of the jaw belonging to the part of the dental arch, the roots of the teeth and/or the gums located in this section. If necessary, the target section can also comprise only one section of the jaw and possibly the associated gums if there are no teeth in this area. The target section may comprise part of a dental arch, e.g. ¼ or ⅓ of a dental arch, a quadrant (i.e. half of a dental arch), an entire dental arch or both dental arches.

The MR measurements are in particular imaging measurements. For example, the MR coil may be suitable for recording high-resolution two-dimensional (2D) or three-dimensional (3D) MR images of the target section. For example, imaging sequences with ultrashort echo time (UTE) can be used for this purpose.

The MR coil has one or more coil conductors which are designed, for example, as copper conductors. Together with one or more capacitive elements (e.g. capacitors), these form a resonant circuit. The resonant circuit may be configured to be resonant at the MR frequency.

Depending on the field strength of the main magnetic field of the MRI device, the resonance frequency of the resonant circuit may be between 5 and 300 MHz. Typical frequencies are 23 MHz (0.5 T), 63 MHz (1.5 T), 123 MHz (3 T) and 300 MHz (7 T). Typically, an MR coil is resonant at a certain MR frequency. However, it is also possible to make the MR coil adaptable to different main magnetic field strengths by using an adjustable capacitor the capacitance of which can be adjusted between 10 and 100 pF, for example.

The MR coil has a central part which may be configured to be placed on the occlusal surfaces of the teeth in the target section of the dental arch. The central part may be flat and elongated along the row of teeth. It may be between 5 and 20 mm, such as 8-12 mm wide. The central part may have the shape of a horseshoe or part of a horseshoe. Due to the curved shape of the horseshoe, the MR coil may be adapted to the shape of the target section. The central part may be between 3 and 20 cm long, such as between 5 and 15 cm, in order for example, to cover a quadrant of the jaw or the entire lower or upper dental arch.

The two longitudinal sides of the central part are adjoined by the two legs, which may project at an angle of 70°-130° from the plane of the central part, which during use runs approximately parallel to the occlusal plane. In this way, the two legs encompass the teeth and, if necessary, the associated jaw in such a way that one leg is arranged on the inside and one leg on the outside of the dental arch. The inside is understood here as the side facing the oral cavity, and the outside as the side facing the check. The coil conductors of the resonant circuit arranged in the legs are therefore very close to the roots of the teeth, so that a very high signal-to-noise ratio of the MR measurements, in particular the imaging MR measurements, can be achieved in this area. This means that the SNR of the entire tooth structure, including the roots of the teeth, the gums and jaw bones, can be increased many times over.

The MR coil may be configured to couple inductively with an MR receiver coil. This allows the receiving field of the MR receiver coil to be amplified, which increases the SNR of the MR measurement. This has the advantage that the MR coil does not have to be connected to the MRI device via cables. The MR coil thus increases the SNR locally so that the area of interest in the target section is better visible on the MR images. The MR receiver coil is, for example, the body coil integrated into the MRI device. In an exemplary embodiment, however, it is a local coil, for example, a head coil.

According to one embodiment, the target section may comprise at least 4 teeth of a dental arch, such as at least 5 teeth, or such as at least the majority of a quadrant of the jaw, or such as at least the majority of two quadrants of a lower or upper jaw. In contrast to the RF coil of DE 10 2014 220 116 A1, the MR coil thus allows an MR measurement on a larger section of the dental arch or jaw. In particular, the MR coil according to this embodiment allows imaging of an entire quadrant of the jaw, the lower jaw or upper jaw, or even imaging of the entire jaw. In this case, the MR coil can be designed in such a way that the longitudinal sides of the central part are joined on the outside and inside by legs which protrude upwards and downwards. In this embodiment, the MR coil may be designed like a dental brace for the lower and upper jaw together.

According to one embodiment, the coil conductors comprise at least one butterfly coil each comprising at least two wings, which are arranged in the two legs of the MR coil in such a way that at least two wings lie opposite one another on the inside and outside of the dental arch and are connected to one another, in particular crosswise, in the area of the central part. In this context, a butterfly coil is understood to be an MR coil comprising two wings, which in this case are arranged in the two legs of the MR coil. In the case of a butterfly coil, the current paths may intersect between the two wings. In this case, the coil conductors may intersect in the area of the central part. In particular, the butterfly coil can have one or more turns in each of the two wings, which thus lie opposite one another on the inside and outside of the dental arch, and which are connected to one another crosswise in the area of the central part. The proposed design is advantageous because the main field of the resonant circuit is then parallel to the occlusal plane, and thus a patient lying normally in a typical MRI device is in the xy-plane. In a typical MRI device, the direction of the main magnetic field is horizontal, aligned in the longitudinal direction of the patient tunnel. It is therefore advantageous if MR coils for detecting the MR signal can detect a B1 field perpendicular to the z-direction particularly well. This is the case with the MR coil according to the disclosure in this embodiment. Advantageously, the turns running in the two legs are coupled to one another in the central part, for example, conductively connected to one another, such as crosswise. This ensures that the magnetic field generated by the resonant circuit in the two wings/legs is mutually amplified.

According to one embodiment, the target section of the MR coil may comprise at least two quadrants of a lower or upper jaw, the coil conductors comprising one butterfly coil per quadrant of the jaw. This has the advantage that, on the one hand, an entire dental arch or an entire half of the jaw (lower jaw or upper jaw) lies in the sensitive area of the MR coil. The magnetic field of the two butterfly coils can be advantageously amplified by the structure of two butterfly coils as the two butterfly coils are arranged on the right and left side of the jaw during use. By appropriately switching the butterfly coils, they can operate in a constructive mode, an in-phase field being generated in both quadrants of the jaw. In this mode, the B1 field of the MR coil is constructively amplified in both quadrants of the jaw. This enables a particularly high SNR with a simultaneously large sensitive area of the MR coil. Such an MR coil is also referred to here as a “double quadrant coil.” In one embodiment, two double quadrant coils can be combined with one another for the upper jaw and lower jaw in order to record the entire jaw at the same time.

According to one embodiment, the two butterfly coils are coupled to one another, in particular via a conductor section, in such a way that the B1 field generated by the two butterfly coils is superimposed constructively. This has the aforementioned advantage of a particularly high SNR in the entire lower jaw or upper jaw. Coupling can be achieved, for example, by connecting the two butterfly coils crosswise to one another at a position which is in front of the front teeth during use. This creates a single coil with a correctly oriented B1 field.

According to an alternative embodiment, the two butterfly coils are independent, i.e. not connected to one another via a conductor, but they couple with one another inductively. This leads to a splitting into two modes, only one mode generating an in-phase field in both quadrants of the jaw. In an exemplary embodiment, the resonance in which the B1 field in the jaw is constructively amplified is stimulated.

According to one embodiment, the coil conductors comprise flat conductor tracks, in particular copper tracks, which are applied to an insulating substrate. This embodiment is advantageous as it allows the MR coil to be produced in any shape. The substrate can be made of any insulating material, in particular a non-conductive or electrically insulating material, for example synthetic resin, e.g. epoxy resin, plastic, rubber or Plexiglas. For example, the substrate can have the shape of a dental brace. The conductor tracks are then applied to this. This makes it possible to produce the MR coil in an advantageous shape which may be adapted to the shape of the dentition. The substrate may be flexible to a certain extent, for example by up to 10 degrees, in order to adapt to different dentition shapes. The conductor tracks may, for example, have a width of 1 to 5 mm and a thickness of 5-100 μm. In an exemplary embodiment, all coil conductors are designed as flat conductor tracks.

The MR coil can advantageously be covered or coated with a tissue-friendly material (dental material), for example with a plastic, synthetic resin or rubber. This allows the MR coil to be worn comfortably in the mouth like a dental brace. Advantageously, the material may be suitable for sterilization so that the MR coil can be used—after appropriate sterilizat—on-by different patients.

According to one embodiment, a capacitance of the at least one resonant circuit may be formed by two coil conductors of the at least one resonant circuit running essentially parallel to one another, at least in sections, a dielectric being arranged between the two coil conductors. The dielectric may be, for example, the insulating substrate described above. This embodiment has the advantage that no or fewer capacitors are required, leading to cost savings. In an exemplary embodiment, the MR coil does not contain any additional components in the form of capacitors. This makes the MR coil more reliable as there are fewer solder joints or components which can be damaged. In an exemplary embodiment, two coil conductors run at least substantially parallel to one another in sections in that they are applied to opposite sides of an insulating substrate. The uniform thickness of the substrate ensures an essentially constant distance between the conductor tracks.

“Essentially” can be understood to mean a deviation of up to ±15%, such as up to ±10%.

According to one embodiment, the at least one resonant circuit may be configured as a Transmission Line Resonator (TLR). When constructed as a TLR, the coil conductor may be arranged in such a way that it simultaneously represents the inductances and the capacitance in order to form a resonant circuit. On the one hand, this has the advantage that no discrete capacitors are required, which take up additional space in the mouth and may have to be elaborately coated in plastic. It is also less expensive to produce as no additional components need to be used. Finally, the design is more robust as fewer fault-prone solder joints are required. The resonance frequency may be determined by the geometry, in particular the size of the TLR, the conductor width, the conductor length, the thickness of the dielectric between the conductors, etc. A TLR design is easier to implement for higher frequencies (≥123 MHz) but is also possible at 23 MHz (0.5 T). At low frequencies (in particular at ≤63.5 MHz), a multi-turn design may be used to achieve the resonance frequency. A TLR with several turns is also referred to as a Multi-Turn Transmission Line Resonator (MTLR).

According to one advantageous embodiment, the resonant circuit has at least one abrupt change in impedance, in particular an interruption. Such an abrupt change in impedance in a transmission line or a coil conductor causes a reflection of the transmitted signal, and two such reflectors cause a standing wave between them. The resonance frequency may be determined, among other things, by the distance between the two abrupt impedance changes and the effective dielectric constant of the transmission line. In this embodiment, the resonant circuit can also be designed as a TLR.

According to one embodiment, the at least one resonant circuit may comprise at least two interrupted conductor rings which run essentially parallel to one another, a dielectric being arranged between the two conductor rings. This is an advantageous embodiment of a TLR. For example, coil conductors may be arranged on opposite sides of a flat dielectric, for example as conductor tracks on opposite sides of a flat substrate. This embodiment combines the advantages described above of a resonant circuit with or without fewer additional components in the form of capacitors with the advantages of using coil conductors which are applied to a substrate, for example as flat conductor tracks, in particular made of copper. At the interruptions, the current is naturally equal to 0, so that a standing wave with a current node and a voltage antinode at the interruptions is created in the resonant circuits. The interruptions of the interrupted conductor rings are in particular sections in which the coil conductors are not continuous but have a gap. The gap can be, for example, 0.1-2 mm in size. Advantageously, the at least two interrupted conductor rings each have one or more interruptions, e.g. 1-5 interruptions. The interruptions can be located directly opposite each other on the two sides of the dielectric, but they can also be arranged offset to one another along the conductor ring.

According to one embodiment, the at least two interrupted conductor rings are each designed as butterfly coils, the conductor rings each having at least two turns, in each case at least one turn extending in one leg of the MR coil. The TLR thus forms a butterfly coil. As described above, this is advantageously arranged in the arms of the MR coil in such a way that an entire quadrant of the jaw can be viewed through a butterfly coil. A TLR butterfly coil therefore has two conductor rings, each of which has at least one turn in one leg and at least one turn in the other leg of the MR coil. In each leg/wing at least two conductor rings thus run essentially parallel to one another, in particular on both sides of a dielectric substrate or carrier material.

According to one embodiment, in this embodiment the interruptions of the conductor rings are arranged in the area of the central part. In particular, this is in the area of the crossover. It has been shown that through the formation of the voltage antinode at this point, the field enhancement or the B1 field is particularly advantageous for the purpose of MR imaging in teeth and jaws. However, depending on the resonance frequency, other embodiments are also possible in which the interruptions are arranged at other points on the conductor rings. There can also be 2 or 3 or more (e.g. up to 5) interruptions per conductor ring, which are distributed, for example, around the conductor ring.

According to one embodiment, the interrupted conductor rings have more than two turns, the conductor rings in each leg of the MR coil having two or more turns in particular. A design with more than one turn increases the capacitance and the inductances of the resonant circuit, whereby the resonance frequency is lowered. Thus, a design with more than one turn allows the use of the MR coil according to the disclosure at lower magnetic field strengths, for example, at 23 MHz (0.5 T) and below.

According to one embodiment, the resonance frequency of the MR coil can be changed. In an exemplary embodiment, it is possible to decouple the MR coil from the respective transmitter coil of the MRI device used while the radio frequency pulses (RF pulses) necessary for the MR measurement are being transmitted. This is advantageous in order to avoid heating of the MR coil and distortion of the transmission field. This can be advantageously achieved by placing two antiparallel diodes parallel to a capacitor of the resonant circuit. In the case of a TLR, such antiparallel diodes can also be connected via the interruption. The diodes act like a passive switch, which bypasses the capacitor in the event of a high induced voltage, as is to be expected during the RF transmission pulse. As a result, the resonant circuit is no longer resonant at the MR frequency at this moment. At low voltage, the capacitor is no longer bypassed by the diodes, the MR coil then has the set resonance frequency. As an alternative to the diodes in parallel with the capacitor, an additional resonant circuit is also conceivable as a “blocking circuit,” as is used for “detuning” MR receiver coils. However, this would take up more space.

According to one embodiment, the MR coil may comprise a covering surrounding the coil conductors, which is particularly flexible in order to adapt to different jaw geometries. The covering may be made of a suitable material approved for use in the oral area (dental material), e.g. made of a suitable plastic, rubber or gum. It allows a patient to place the MR coil in the mouth and, if necessary, bite on it to hold the MR coil in position. Furthermore, the covering advantageously results in the MR coil being more comfortable to wear.

The disclosure is also directed towards an MR coil assembly comprising an MR coil (e.g., according to one or more aspects) and a transducer coil inductively coupled thereto. The transducer coil may be a “conventional” MR receiver coil or an MR-transmit-receive coil of an MRI device, for example a body coil, or advantageously a head coil or another local coil of an MRI device. The local coil can be, for example, a surface coil or a phased array coil. However, the transducer coil can also be a dedicated coil which has been specially produced for the purpose of receiving the signal from the intraoral coil according to the disclosure and, if necessary, transmitting it to the MRI device. Thus, the MR coil itself advantageously does not require any electronics for the receiving chain. Rather, the MR coil may merely contain the conductor tracks and possibly discrete passive components, as well as generally a carrier material/substrate and/or a covering. A high level of patient comfort also results as the mouth can be closed again for the measurement.

Finally, the disclosure is also directed towards an MRI device which has an MR coil or an MR coil assembly according to the disclosure.

Furthermore, the disclosure is directed towards the use of an MR coil, as described here, for intraoral MR measurements on a target section in the dental area.

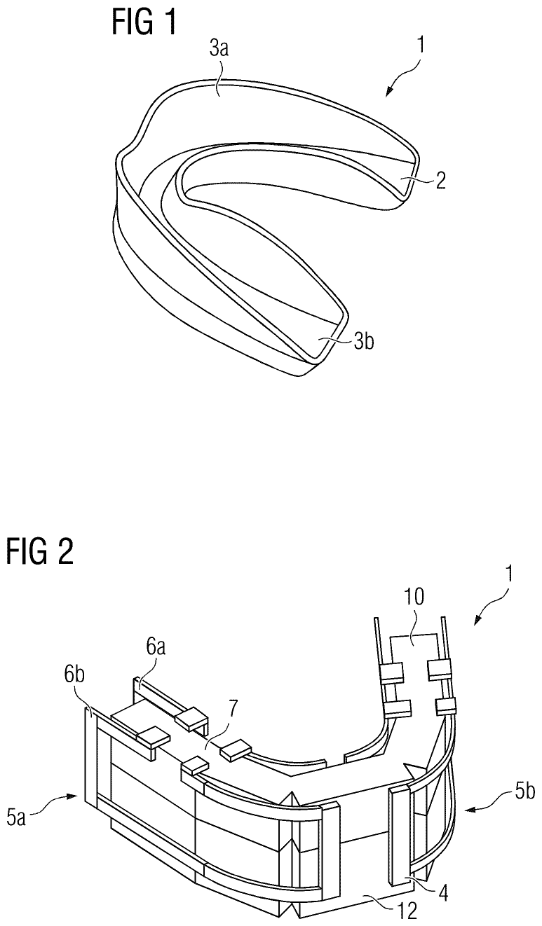

FIG. 1 shows the basic shape of an MR coil 1 according to an embodiment of the disclosure. In this embodiment, the target section may essentially comprise an entire dental arch, possibly including the gums and associated lower jaw or upper jaw. The MR coil 1 has the basic shape of a dental brace. In particular, it has a flat, horseshoe-shaped curved central part 2. One leg protrudes from the central part on the inside and one on the outside. The leg on the outside is referred to as 3a, the leg on the inside as 3b. The MR coil can be placed on the dental arch in such a way that the two legs 3a, 3b encompass the dental arch and come to rest near the inner surfaces of the tooth or jaw (leg 3b) and the outer surfaces of the tooth or jaw (leg 3a). The coil conductors are not shown in FIG. 1; they run inside the MR coil, including the legs.

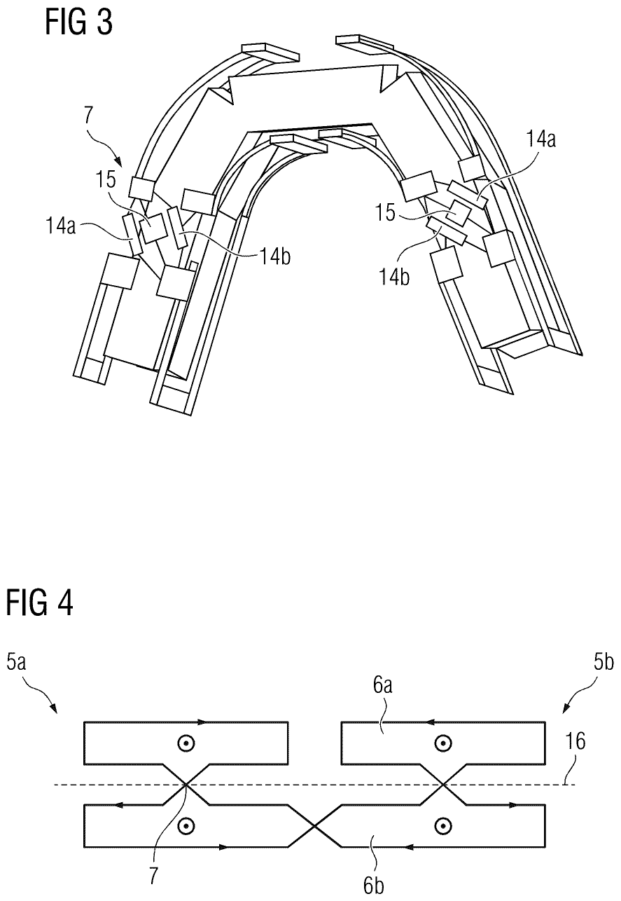

FIG. 2 shows the course of the coil conductors 4 according to an embodiment of the disclosure, namely the so-called “double quadrant coil.” The MR coil 1 may be mounted on a lower jaw with a dental arch 10 and underlying gums 12 and jaw, as would be done for an MR measurement. In this case, the coil conductors 4 of the MR coil comprise two butterfly coils 5a and 5b. Each of these butterfly coils consists of one turn 6a on the inside of the dental arch and one turn 6b on the outside of the dental arch. These turns run in the legs 3a, 3b of the MR coil 1. On the occlusal surface, i.e. in the area of the central part 2, the two turns 6a and 6b are coupled to one another, as shown in more detail in FIG. 3.

Advantageously, the two turns 6a and 6b of a butterfly coil 5a or 5b are cross-connected at the crossing point 7. In the example shown in FIG. 3, this is done by means of a typically soldered conductive connection and a capacitor 15. The capacitor 15 provides the necessary capacitance to form a resonant circuit from the coil conductors 4. The size of the capacitor 15 may be selected such that the resonant circuit is resonant at the MR frequency. In this embodiment, two oppositely directed diodes 14a and 14b are connected in parallel to the capacitor 15. These close at high voltages, such as occur during the transmission of the RF pulses for the MR measurement. This “detunes” the resonant circuit so that it is no longer resonant at the MR frequency. This has the advantage that the MR coil is then decoupled from the transmitter coil and the transmission field is not amplified or distorted. Both butterfly coils 5a, 5b are connected to the capacitor 15 and the diodes 14a, 14b in the same way at the crossing point 7.

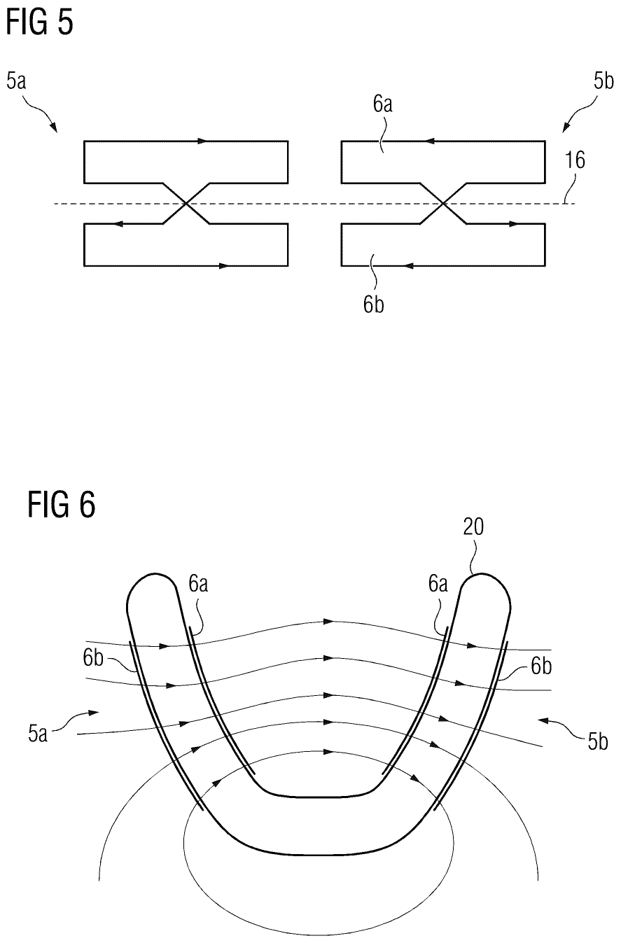

FIG. 4 shows a first way in which the two butterfly coils 5a, 5b can be coupled. In the example shown, the two butterfly coils 5a, 5b are conductively connected to one another crosswise in front of the teeth so that a single resonant circuit with a correctly oriented B1 field is created. The current course can be seen from the B field lines of FIG. 6. In particular, FIG. 6 shows that the B1 field of the two butterfly coils is amplified in the area of the jaw 20 if the circuit is correctly connected. In this way, a large increase in the SNR in the area of interest of the teeth and roots of the teeth is possible.

FIG. 5 shows a second way in which the two butterfly coils 5a, 5b can be coupled to one another. In this case, they are only coupled to one another inductively. In other words, the MR coil may be used to attach two independent butterfly coils 5a, 5b with crossing points near the occlusal surfaces of the teeth, each via a quadrant of the jaw. Thus, the two coils couple inductively not only with a receiver coil, but also with each other. This leads to a split into two modes, with only one mode generating an in-phase field in both quadrants of the jaw. The resonance mode which constructively amplifies the B1 field in the jaw is selected, as shown in FIG. 6.

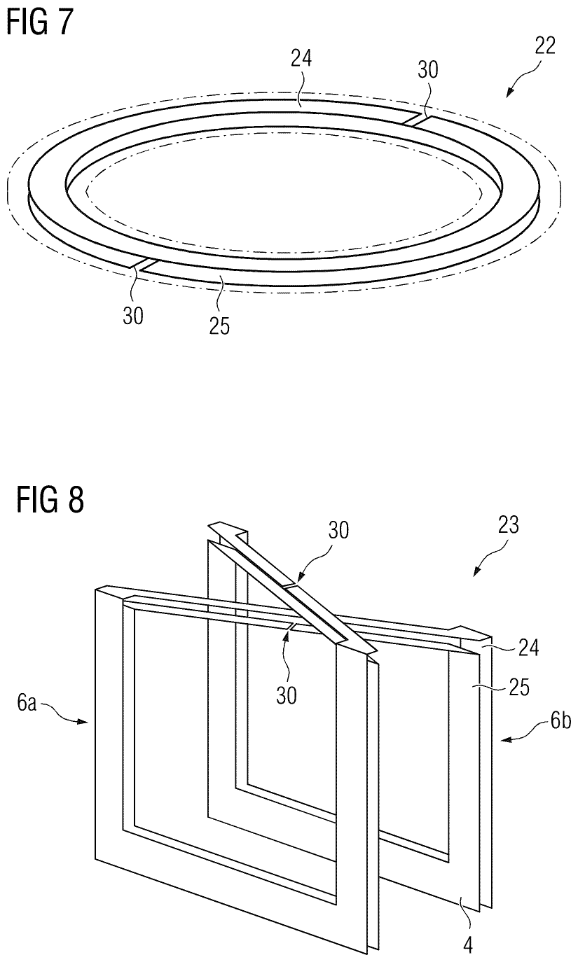

FIG. 7 shows a schematic view of a simple Transmission Line Resonator (TLR) 22. This consists of two superimposed conductor rings 24, 25, between which a dielectric is arranged, for example an insulating substrate. The opposing conductors 24, 25 act like a capacitance, so a discrete capacitor is no longer necessary to form a resonant circuit. At 30, the two conductor rings 24, 25 each have an interruption.

FIG. 8 shows a schematic view of a butterfly TLR 23, which is suitable for placing on a quadrant of the jaw and thus being part of a coil conductor 4 of an MR coil 1 according to an embodiment of the disclosure. For better visibility of the cable routing, the insulating material between the two layers of the TLR is hidden and only the coil conductors 4 are shown. The TLR 23 has the shape of a butterfly coil with two wings 6a and 6b, which are each formed by one turn and which lie opposite one another on the inside and the outside of a row of teeth 10. The butterfly coil may comprise two interrupted conductor rings 24, 25, which run essentially parallel to one another, for example, on two sides of an insulating substrate or carrier material. Discrete capacitors are therefore no longer required. At the crossing point, interruptions 30 of the conductor rings 23, 24 are provided in each case. By varying the width of the conductor tracks 4, the distance between the conductor rings 24, 25 and the number of turns, it is possible to adjust the resonance frequency of the butterfly TLR 23 such that a desired MR frequency is achieved. The conductor tracks 4 are always shown straight here, but this is due to the schematic view. The conductor tracks, like the MR coil, may be adapted to the shape of the sensitive area or target section, that is to say, for example slightly curved with the dental arch.

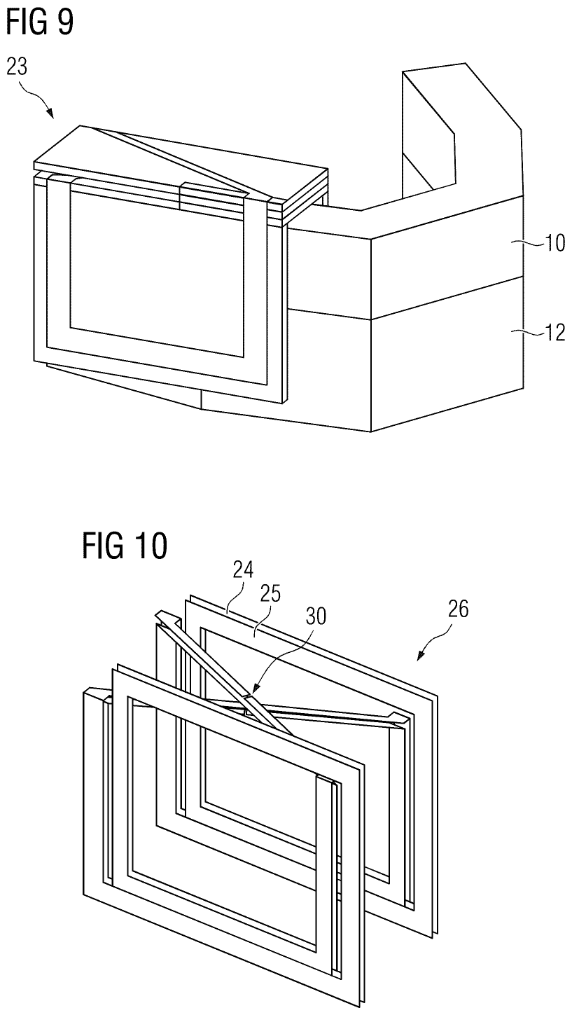

FIG. 9 shows the butterfly TLR 23 of FIG. 8 mounted on a jaw 12 with a dental arch 10.

In an alternative embodiment (not shown), it is also possible to provide more than two interruptions 30. This acts like a series connection of several capacitors. The effective capacitance is reduced as a result and the resonance frequency thus increased.

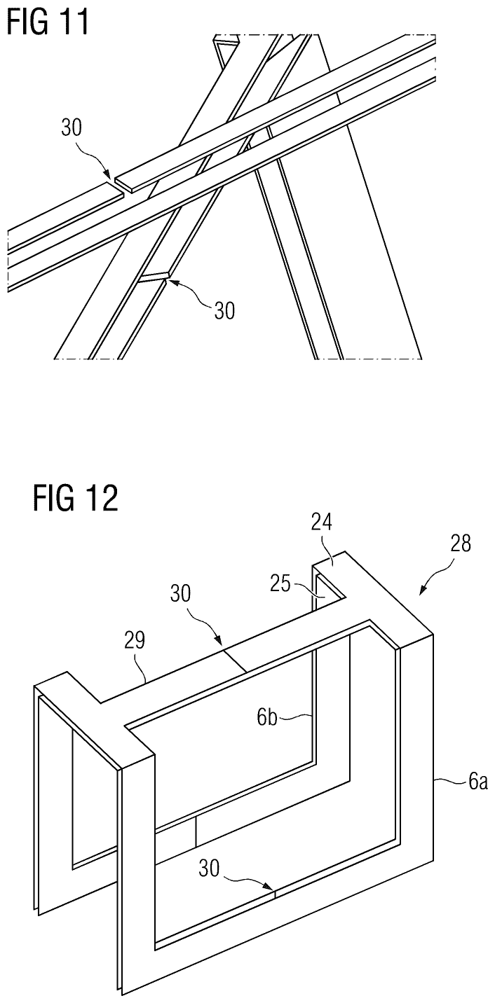

FIG. 10 shows another embodiment of a butterfly TLR 26, which has a similar structure to the butterfly TLR 23 of FIG. 8, but with two turns provided in each leg of the MR coil 1. The turns run concentrically so that the conductor tracks can be arranged on a single substrate. Each of the two turns is in turn duplicated with two conductor rings 24, 25 running parallel to one another. In the area of the central part, the turns of the butterfly coil 26 are connected to one another on the two sides of the dental arch. The interruption and 30 are also arranged here. This is shown again enlarged in FIG. 11.

FIG. 12 shows yet another embodiment of a butterfly TLR 28. In this embodiment, the butterfly TLR has a common center bar 29. In this embodiment, three interruptions 30 are required, one on the center bar 29 and one on each of the two wings 6a, 6b. More interruptions are also conceivable. The interruptions 30 on the center bar 29, on the one hand, and the two wings, on the other hand, are advantageously arranged in opposite conductor rings or layers of the TLR. In the illustration of FIG. 12, the interruptions 30 in the wings are on the lower conductor ring, while the interruption 30 on the center bar 29 is located on the upper conductor ring.

A prototype of an intraoral MR coil according to an embodiment of FIGS. 2 and 3 was produced, and it was found that it could be used to perform MR imaging with very high SNR on the dental arch. The intraoral dental coil according to the disclosure significantly improved the SNR compared to extraoral coils. It can cover an entire quadrant of the jaw, an entire half of the jaw or even the entire jaw. The MR coil according to the disclosure has a large penetration depth which extends into the jawbone and over the entire roots of the teeth. No cables are required, which makes the coils very easy to handle and much more comfortable for patients. The fact that the MR coil is realized entirely without active components also makes it cost-effective to realize.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

1. A magnetic resonance (MR) coil for intraoral MR measurements on a target section in a dental area, the target section including at least part of a dental arch, the MR coil comprising:
coil conductors configured to form at least one resonant circuit;
a central part; and
two legs protruding from the central part in which the coil conductors of the at least one resonant circuit are arranged,
wherein the MR coil is configured to be placed on the dental arch such that the two legs encompass the dental arch in the target section such that a first leg of the two legs is arranged on an inside of the dental arch and a second leg of the two legs is arranged on an outside of the dental arch, and
wherein the MR coil has a shape adapted to the shape of the target section.

2. The MR coil as claimed in claim 1, wherein the target section comprises at least 4 teeth of the dental arch, at least a majority of a quadrant of a jaw, or at least a majority of two quadrants of a lower or upper jaw.

3. The MR coil as claimed in claim 1, wherein the coil conductors comprise at least one butterfly coil including at least two wings arranged in the two legs of the MR coil such that the at least two wings lie opposite one another on the inside and the outside of the dental arch and are coupled to one another in an area of the central part.

4. The MR coil as claimed in claim 3, wherein the target section of the MR coil comprises at least two quadrants of a lower or upper jaw, and wherein the coil conductors comprise one butterfly coil per quadrant of the jaw.

5. The MR coil as claimed in claim 4, wherein the two butterfly coils are coupled to one another such that a B1 field generated by the two butterfly coils is superimposed constructively.

6. The MR coil as claimed in claim 1, wherein the coil conductors comprise flat conductor tracks applied to an insulating substrate.

7. The MR coil as claimed in claim 1, wherein a capacitance of the at least one resonant circuit is formed by two coil conductors of the at least one resonant circuit running parallel to one another, at least in sections, a dielectric being arranged between the two coil conductors.

8. The MR coil as claimed in claim 1, wherein the at least one resonant circuit is configured as a Transmission Line Resonator.

9. The MR coil as claimed in claim 8, wherein the at least one resonant circuit comprises at least two interrupted conductor rings extending parallel to one another, a dielectric being arranged between the two interrupted conductor rings.

10. The MR coil as claimed in claim 9, wherein the at least two interrupted conductor rings are each configured as butterfly coils, and wherein the at least two interrupted conductor rings each have at least two turns, of which at least one turn extends in one of the two legs of the MR coil in each case.

11. The MR coil as claimed in claim 9, wherein the at least two interrupted conductor rings each have one or more interruptions.

12. The MR coil as claimed in claim 9, wherein the at least two interrupted conductor rings each have two or more turns and are each disposed in one of the two legs of the MR coil.

13. The MR coil as claimed in claim 1, wherein the MR coil is configured to inductively couple with a transducer coil.

14. The MR coil as claimed in claim 1, further comprising a covering configured to surround the coil conductors, the covering being flexible to adapt to one or more different jaw geometries.

15. The MR coil as claimed in claim 1, wherein the MR coil is further configured to be placed on the dental arch such that the two legs encompass at least a portion of a jaw in the target section.

16. A magnetic resonance (MR) coil assembly, comprising:
an MR coil as claimed in claim 1; and
a transducer coil configured to be inductively coupled thereto.
